(19) United States
(12) Patent Application Publication
(10) Pub. No.: US 2004/0034481 A1
(43) Pub. Date: Feb. 19, 2004

(54) Methods for identifying a prospective binding site on a target molecule and for characterizing a site on a target molecule

(76) Inventor: John R. Hurst, San Diego, CA (US)

Correspondence Address: Philip D. Reilly, Morrison & Foerster LLP, 755 Page Mill Road, Palo Alto, CA, 94304-1018, US

(21) Appl. No.: 10/222,452

(22) Filed: Aug. 16, 2002

(51) Int. Cl. G06F 19/00; G01N 33/48; G01N 15/06

(52) U.S. Cl. 702/27; 702/19; 422/68.1

(57) ABSTRACT

Methods which may be implemented in computer systems for characterizing sites on a target molecule, for identifying prospective binding sites on a target molecule, and for identifying a position for a candidate molecule in a computation involving the target molecule and candidate molecule.

### FIELD OF THE INVENTION

Methods and systems described in this patent generally relate to methods and systems for characterizing sites on a target molecule and identifying prospective binding sites on a target molecule. Methods described in this patent may be implemented in a computer system and may be embodied as computer programs in an article of manufacture.

### BACKGROUND OF THE INVENTION

Drug discovery may be conceptually understood in two steps: 1) target identification and 2) lead identification and optimization. In the first step of target identification, a large molecule, which may be but is not limited to a cell-surface receptor or intra-cellular protein, referred to as the target, may be identified with a particular biological pathway or structure of interest. Once a potential target has been identified, it must be screened against a large number of small molecules to determine whether the target is drugable—i.e. whether any small molecules can be found to bind or interact with the target. The second step of lead identification and optimization is very expensive and time consuming, often involving experimentally screening a target against many millions of small molecules to determine binding affinities. Once one or more drug leads to a target have been identified, it is still often necessary to optimize a lead in order to improve its pharmacological efficacy. In this step, referred to as lead optimization, synthetic chemists chemically modify the lead in order to increase or decrease its binding to the target, modify its susceptibility to degradative pathways, or modify its pharmacokinetics.

The current combinatorial methods of lead identification and to a lesser extent, lead optimization, could be improved if rationalized. Rational lead optimization, as used herein, refers to using theoretical methods to determine a set of likely lead candidates to a given target before experimentally screening lead-target binding. Rational lead optimization offers the possibility of reducing drug discovery time and expenses by reducing the number of potential lead-target screens.

Early methods of rational lead optimization sought to find a set of potential leads from a database of small molecules chemically similar to a known lead. The earliest of these techniques was performed by the synthetic chemist in the process of producing the next substance to test. The chemist would make a set of small changes to the structure and determine if those changes had a beneficial or detrimental effect on the efficacy. This process, called analog synthesis, is effective, although time-consuming and expensive. Analog synthesis is the basis for medicinal chemistry, and remains an important part of drug discovery today.

Early attempts at using computers to make the lead optimization process more efficient involved determining chemical similarity between a potential lead and a known lead using graph-theoretical treatments. Methods of this type include searching a database of compounds for those that contain the same core structure as the lead compound—called substructure searching or two-dimensional (2-D) searching—and searching for compounds that are generally similar based on the presence of a large number of common fragments between the potential lead expansion compound and the lead compound—called 2-D similarity searching. These techniques are somewhat effective, but are limited to finding compounds that are obviously similar to the lead, thus affording the medicinal chemist few new insights for directing the synthesis project.

Another technique using computers makes use of the knowledge of how the small molecules bind to the large molecule. There are often special groups called pharmacophore groups that are responsible for most of the stabilization energy of the complex. The three-dimensional (3-D) searching techniques try to find from a large database of potential lead compounds those that have essentially the same geometrical arrangement of pharmacophore groups as the lead compound. The 3-D arrangement of the pharmacophore groups required for interaction with the large molecule is called a pharmacophore model or 3-D query. Compounds that are found to contain the correct arrangement are called “hits,” and are candidates for screening. These hits differ from the substructure or 2-D similarity hits in that the backbone of the structure may be quite different from that of the original lead compound, and often represents an important new area of chemistry to be explored.

The simplest of the 3-D searching techniques compares the position and arrangement of the pharmacophore groups of 3-D structures as stored in the database. This is referred to as static 3-D searching. A fairly obvious shortcoming to determining chemical similarity from a database of small molecules of fixed geometry is that molecules often do not have rigid structures, but rather have a large number of accessible conformations formed from rotating the molecular framework of a molecule about its single bonds. These bonds that can easily be rotated are referred to as rotatable bonds. As used herein, molecular configurations defined by rotations about the dihedral angles of its rotatable bonds will be referred to as rotomers. Small molecules in pharmaceutical databases typically contain an average of six to eight rotatable bonds per molecule. This can easily afford a set of accessible rotomers that number in the millions. Searching just one static conformation (rotomer) from among millions of possible rotomers for the correct arrangement of groups will cause many compounds that could be good leads to be missed.

In order to consider energetically accessible rotomers, early small molecule databases often contained a small subset of the accessible rotomers of each small molecule. Herein, this technique is called multi-conformational 3-D searching. However, attempting to define individually each rotomer for a given small molecule quickly becomes impractical as the number of atoms in a given small molecule increases. Accordingly, other early methods of searching small molecule databases for potential target leads attempted to define a conformational space for each small molecule. See U.S. Pat. No. 5,752,019.

A further extension of 3-D search technology involves investigation of the accessible conformational space of the potential hits as part of the searching process. These techniques, called conformationally flexible 3-D searching techniques, adjust the conformation of the potential hit according to the requirements of the 3-D pharmacophore query. One such technique that has been found to be effective uses a method called “Directed Tweak”. See Hurst, T. Flexible 3D Searching: The Directed Tweak Technique, 34, J. Chem. Inf. Comput. Sci. 190 (1994). This method is very effective for finding molecules of interest when the geometry of the binding site of the large molecule is not known. Directed Tweak adjusts the conformation of the small molecule by changing the angle values of the rotatable bonds. This method therefore ignores changes in conformation because of bond stretching and bond bending. Bond stretching vibrations for molecules near room temperature typically change the length of a bond by about 0.05 Angstroms (Å). Bond bending between three connected atoms typically moves one of the atoms by about 0.1 Å. Rotation about rotatable bonds often moves atoms by several Ås or tens of Ås. Thus, adjusting only the rotatable bond values includes essentially all of the accessible conformational flexibility of a small molecule.

When the binding site of the target protein is known, another way to identify potential leads is by docking potential leads into the binding site. Docking approaches can be classified based on how they characterize the ligand-binding site of the protein. Grid-search techniques fill the space around the binding site with a 3-D grid, precompute the potentials (van de Waals, electrostatic, etc.) at each grid point without a ligand, then sample different ligand conformations and orientations on the grid and compute the resulting binding energy.

Early attempts of rational lead optimization modeled small molecule docking to a target using molecular mechanics force field methodologies. Molecular mechanics methodologies (also called force field methodologies) attempt to model the short range and long range forces between a target and a small molecule using field representations. U.S. Pat. No. 5,866,343, treats the target and small molecule using separate potential energy fields. Each potential energy field comprises a 1/R long range electrostatic contribution and a short range 6-12 Lennard-Jones contribution. The interaction energy between a target and a small molecule is then calculated for the position of the small molecule relative to the large one. The position is then adjusted iteratively so as to give lower and lower energies. This continues until a low energy arrangement is found. Another example is AutoDock (Morris, G. M., Goodsell, D. S., Halliday, R. S., Huey, R., Hart, W. E., Belew, R. K. and Olson, A. J. “Automated Docking Using a Lamarckian Genetic Algorithm and Empirical Binding Free Energy Function”, J. Computational Chemistry, 19 (1998), 1639-1662.) which uses simulated annealing in its previous releases, but now applies a hybrid genetic algorithm to sample over the feasible binding nodes of the ligand relative to the protein. The advantages of grid-based docking are that a template of favorable interactions in the ligand-binding site does not need to be defined, thus reducing bias in modeling the protein-ligand interactions, and evaluation of binding modes is made more efficient by precomputing the potential energy that results from interaction with the protein at each point on the grid. However, the accuracy and timing of this approach depends on the grid fineness, making this approach too computationally intensive for database screening, in which thousands of molecules (as well as ligand orientations and shapes, or conformers) need to be tested. Furthermore, precomputation of the protein grid potentials limits this approach to rigid binding sites.

When a ligand-binding site in a protein is known, a grid template may be used by constructing a template for ligand binding based upon favorable interaction points in the binding site. During the search for a favorable ligand-binding mode, different conformations of the ligand can be generated and subsets of its atoms matched to complementary template points as a basis for docking the ligand into the binding site. One of the principal advantages to a template-based approach is that a template can incorporate known features of ligand binding (for example, conserved interactions observed experimentally for known ligands). Thus, the docking search space is reduced to matching N ligand atoms onto N template points, rather than the six-dimensional orientational search space (three degrees of rotational freedom and three degrees of translational freedom) required in other approaches for sampling and evaluating ligand binding.

Another well-known docking tool is DOCK (Schoichet, B. K., Bodian, D. L., and Kuntz, I. D., J. Comput. Chem. 13 (1992) 380). In this program the template typically consists of up to 100 spheres that generate a negative image of the binding site. During the search, subsets of ligand atoms are matched to spheres, based on the distances between ligand atoms. DOCK has been extended to consider chemistry and include hydrogen-bonding interaction centers in addition to the shape template. Other current template approaches specify a set of interaction points defining favorable positions for placing polar ligand atoms or hydrophobic (nonpolar) centers, e.g., aromatic rings. Such a template can be generated automatically, e.g., by placing probe points on the solvent accessible surface of the binding site, or interactively by superimposing known protein-ligand complexes to identify favorable interaction points based on observed binding modes for known ligands. Another docking program, FlexX (licensed by Tripos Inc.—1699 South Hanley Road, St. Louis, Mo. 63144-2913, phone: 314-647-1099) uses a template of 400 to 800 points when docking small molecules (up to 40 atoms, not including hydrogens) to define positions for favorable interactions of hydrogen-bond donors and acceptors, metal ions, aromatic rings, and methyl groups. The ligand is fragmented and incrementally reconstructed in the binding site and matched to template points based on geometric hashing (indexing) techniques, bond torsional flexibility is modeled discretely, and a tree-search algorithm is used to keep the most promising partially constructed ligand conformations during the search. Hammerhead (Welch W, Ruppert J, Jain AN. 1996. Hammerhead: Fast, fully automated docking of flexible ligands into protein binding sites. Chem Biol 3(1996), 449-462.) uses up to 300 hydrogen-bond donor and acceptor and steric (van der Waals interaction) points to define the template, and the ligand is incrementally constructed, as in FlexX. A fragment is docked based on matching ligand atoms and template points with compatible internal distances, similar to the matching algorithm used in DOCK. If a new fragment is positioned closely enough to the partially constructed ligand, the two parts are merged, and the most promising placements kept.

Other successful docking approaches, such as GOLD (G. Jones, P. Willett, R. C. Glen, A. R. Leach & R. Taylor, J. Mol. Biol 267 (1997) 727-74.), and the method of Oshiro et al. (Oshiro C, Kuntz I, Dixon J. Flexible ligand docking using a genetic algorithm. J Comput Aided Mol Des 1995;9:113-130.) use genetic algorithms to sample over possible matches of conformationally flexible ligands to the template. GOLD uses a template based on hydrogen-bond donors and acceptors of the protein and applies a genetic algorithm to sample over all possible combinations of intermolecular hydrogen bonds and ligand conformations. However, a drawback of genetic algorithm approaches, including AutoDock, is the high computation time, especially in comparison to fragment-based docking approaches.

The UNITY 3-D searching system (licensed by Tripos Inc.—1699 South Hanley Road, St. Louis, Mo. 63144-2913, phone: 314-647-1099) has been extended to provide what is essentially a docking tool. In this approach, six parameters corresponding to the six rotational/translational degrees of freedom are added to the rotatable bond list, and these parameters are adjusted to place pharmacophoric groups at the positions giving favorable interactions with the receptor. This method produces acceptable accuracy, but is time consuming because the derivatives needed for the minimization must be calculated numerically. Each derivative calculation requires three or more small adjustments to the geometry of the small molecule.

Another current docking method, SPECITOPE (Volker Schnecke, Craig A. Swanson, Elizabeth D. Getzoff, John A. Tainer, and Leslie A. Kuhn, Screening a Peptidyl Database for Potential Ligands to Proteins with Side-chain Flexibility Proteins: Structure, Function, and Genetics, Vol. 33, No. 1, 1998, 74-87) combines grid methods with adaptive geometry techniques in order to model protein side chain flexibility. SPECITOPE uses a binding site template to limit the orientational search for each prospective ligand and uses distance geometry techniques to avoid the computationally intensive fitting of infeasible ligands into a binding site. The speed gained by distance geometry methods allows the modeling of protein side chain flexibility during docking.

The results of fast docking tools to predict ligand binding modes a priori (i.e. for cases where there is no ligand for which the binding location relative to the protein is known) are less reliable (Dixon, J. S., “Evaluation of the CASP2 Docking”, Proteins: Structure, Function, and Genetics, Supplement-1 (1997), 198-204.). Today, most docking tools model full ligand flexibility, but at least in the faster approaches the binding site is kept rigid. Limited protein side-chain flexibility is exploited by GOLD, which considers rotational flexibility of side chains (Jones et al., J. Mol. Biol., 245, 43-53, 1995), while other approaches use rotomer libraries (Leach, A. R. 1994. Ligand docking to proteins with discrete side-chain flexibility. Journal of Molecular Biology 235:345-356); Jackson, R. M., Gabb, H. A. and Stemberg, M. J. E. 1998. Rapid refinement of protein interfaces incorporating solvation: Application to the docking problem. Journal of Molecular Biology 276:265-285.) Others still are based on molecular dynamics simulations (Wasserman Z R, Hodge C N. 1996. Fitting an inhibitor into the active site of thermolysin: A molecular dynamics study. Proteins 24:227-237.).

Another consideration that influences the accuracy of docking simulations is the solvation of the binding site (Ladbury, J. E. 1996. Just add water! The effect of water on the specificity of protein-ligand binding sites and its potential application to drug design. Chemistry & Biology 3:973-980.). Water bound in the ligand site is known to be a critical determinant of ligand specificity for HIV-1 protease (Lam, P. Y. S.; Jadhav, P. K.; Eyermann, C. J.; Hodge, C. N.; Ru, Y.; Bacheler, L. T.; Meek, J. L.; Otto, M. J.; Rayner, M. M.; Wong, Y. N.; Chang, C. H.; Weber, P. C.; Jackson, D. A.; Sharpe, T. R.; and Erickson-Viitanen, S. 1994. Rational design of potent, bioavailable, nonpeptide cyclic ureas as HIV protease inhibitors. Science 263:380-384.), cholera toxin (Merritt, E. A.; Sarfaty, S.; van den Akker, F.; L'Hoir, C.; Martial, J. A.; and Hol, W. G. 1994, Crystal structure of cholera toxin B-pentamer bound to receptor GM1 pentasaccharide. Protein Science 3(2): 166-175), and other proteins, and is a ubiquitous component in molecular recognition (Raymer, M. L.; Sanschagrin, P. C.; Punch, W. F.; Venkataraman, S.; Goodman, E. D.; and Kuhn, L. A. 1997. Predicting conserved water-mediated and polar Ligand interactions in proteins using a k-nearest-neighbors genetic algorithm. Journal of Molecular Biology 265:445-464). For the docking tool FlexX, a technique called the particle concept has been recently proposed, which adds water molecules at favorable positions during the incremental construction of the ligand in the binding site (Rarey, M.; Kramer, B.; and Lengauer, T. 1999. The particle concept: Placing discrete water molecules during protein-ligand docking predictions. Proteins: Structure, Function, and Genetics 34:17-28.). This method was tested for 200 protein-ligand complexes and the accuracy of the predicted binding modes increased for several cases, including HIV protease. A different approach has been introduced for database screening using DOCK: here, the molecules in the database are solvated, which improves the ranking for known ligands and filters out molecules with inappropriate charge states and sizes in comparison to screening without solvation (Shiochet, B. K.; Leach, A. R.; and Kuntz, I. D. 1999. Ligand solvation in molecular docking. Proteins: Structure, Function, and Genetics 34:4-16.).

In many docking approaches, it may be useful to determine the most likely binding site or sites for molecular docking. When the active site of a protein or other target molecule is not known, an estimation of where to start looking for a molecular docking binding site must be made. When the active site is known, it may still be useful to determine possible allosteric binding sites.

The Dock program (UCSF) and DockIt (Metaphorics (27401 Los Altos #360, Mission Viejo, Calif. 92691, Phone: (949) 367-9091) use a negative image of the receptor site based on filling it with spheres. The latter rates the generated spheres based on their burial score—the most buried spheres are considered more likely to be part of a binding site.

GOLD calculates, for each point potentially in a binding site, the number of times lines through the point intersect the solvent accessible surface of the protein. Points that are deep in pockets, and are thus potential member points of a binding site, will have lines with larger number of receptor intersections than points on the exposed surface of the protein.

The following techniques are examples of methods that have been used for visualizing the surface topology of target molecules.

The SiteID program (Tripos Inc.) displays various properties of a target molecule surface that may relate to the likelihood of the area being an active site. The user can then visualize the structure looking for potential binding sites.

Connolly (Connolly, M, Molecular Surfaces: A Review, http://www.biochem.usyd.edu.au/˜bchurch/netsci.html) has reviewed various procedures and methods for visualizing the surface topology of target molecules, which may assist in the identification of potential binding sites. Badel-Chagmon et al. (Badel-Chagmon, Nessi, J., Buffat, L., Hazout, S., “Iso-depth contour map” of a molecular surface, J. Mol. Graphics, 1994, 12, 162) used the idea of a convex hull (which they define as a wrapping of all planes tangent to an object) in determining the depth of each point on the solvent accessible surface of the protein. This Badel-Chagmon method is a technique for visualizing the molecular relief of the surface a target molecule, and the method consists of producing a visualization of the molecular surface either using a 2-D graph or a 3-D display showing in different colors those portions of a surface at an equal depth from the convex hull surface. This method does not in any way characterize identified sites other than to visually present the depth of portions of the site from a convex hull surface. Because of the specific techniques used in the Badel-Chagmon method for calculating the depth contours, the method does not always yield the correct depth profile.

Edelsbrunner et al. (Edelsbrunner, H, Mucke, E., Three-dimensional alpha shapes, ACM Transactions on Graphics 13, 43-72) used alpha shapes to look for the buried potential binding sites. In this method, Edelsbrunner et al. positioned spheres of a given radius adjacent to the protein. The difference between the alpha shapes of differing radius (alpha value) is used as an indication of potential binding sites. This method is computationally intense, and may miss certain potential sites because of unfortunate selection of alpha values.

To assist in computer simulation of a target and binding molecule (protein-ligand interactions, for example) there is a need for a method that can automatically identify and characterize all prospective binding sites on a target molecule and that can determine initial positions for a binding molecule based on the characteristics of the prospective binding sites.

None of the previous methods described above, however, can determine or characterize in an automatic manner the spatial extent of sites on a target molecule or provide methods for automatically identifying all prospective binding sites on a target molecule. In particular, the method of Dock and Gold require specification of the general area of the binding site, and may not find potential shallow binding sites. The methods of SiteID and the method of Badel-Chagmon assist in visualization of potential sites, but do not allow for automatic characterization. In addition, the previous methods do not allow for information regarding the spatial extent of a prospective binding site to be used to determine placement of a ligand molecule for use in a computer simulation.

### SUMMARY OF THE INVENTION

The inventor has developed new methods for characterizing the spatial extent of sites on a target molecule, identifying prospective binding sites on a target molecule, and determining a position of a candidate molecule relative to a target molecule as a step in a computer simulation. All of the methods described in this patent may be implemented in a computer. In this section we summarize various aspects of these methods and below in the Detailed Description Section we present a more comprehensive description of the methods and their implementation and uses.

One of the methods described in this patent is a method for characterizing the spatial extent of a volume adjacent to a site on a target molecule, and the method includes the steps of: (a) determining a spatial extent surface characteristic of the overall spatial extent of at least a portion of the target molecule; (b) identifying a set of one or more enclosed volumes each (i) being contained within the spatial extent surface, and (ii) being outside of the target molecule; and (c) assigning to one or more of the enclosed volumes in the set one or more descriptors characteristic of the spatial extent of the enclosed volume. This method may further include the step of identifying a position for a candidate molecule relative to the target molecule based on the value or values of the descriptors associated with an enclosed volume. This method may further include the step of characterizing the spatial extent of a site on the target molecule by assigning to the site the one or more descriptors assigned to one or more enclosed volumes adjacent to the site.

Another of the methods described in this patent is a method for identifying a site on a target molecule as a prospective binding site, and the method includes steps (a), (b) and (c) of the method above plus the additional step of identifying a site on the target molecule as a prospective binding site if the descriptors assigned to an enclosed volume adjacent to the site meet one or more criteria. In one version of this method the descriptors may be a descriptor characteristic of the volume of the enclosed volume and the site is identified as a prospective binding site if the volume descriptor is greater than a cutoff value. Specific descriptors that may be used include the volume of the enclosed volume or the volume of a subvolume of the enclosed volume with the same overall shape as the enclosed volume but of smaller dimensions.

Another of the methods described in this patent is a method for identifying a site on a target molecule as a prospective binding site, and the method includes the steps of (a) identifying a set of one or more enclosed volumes, each enclosed volume (i) being outside of a target molecule, and (ii) meeting one or more criteria characteristic of the overall spatial extent of the target molecule; (b) assigning to an enclosed volume one or more descriptors characteristic of the spatial extent of the enclosed volume; and (c) identifying a site on the target molecule as a prospective binding site if the one or more descriptors assigned to an enclosed volume adjacent to the site meet one or more criteria. In one version of these methods, an enclosed volume meets the one or more criteria characteristic of the overall spatial extent of the target molecule if points in the enclosed volume are within a surface characteristic of the overall spatial extent of the target molecule.

Another of the methods described in this patent is a method for determining a position of a candidate molecule relative to a target molecule for use in a computer simulation, and the method includes the steps of (a) identifying a prospective binding site on a target molecule characterized by one or more descriptors; and (b) identifying a position for a candidate molecule relative to the target molecule determined by the value or values of the one or more descriptors. In specific versions of these methods, the prospective binding site may be identified using one of the methods described above.

Another of the methods described in this patent is a method for identifying a set of points indicating a prospective binding site on a target molecule, and the method includes the steps of (a) determining a convex hull surface for a target molecule; (b) identifying a set of points that are inside the convex hull surface and outside of the target molecule; (c) grouping the set of points identified in step (b) into one or more subsets; and (d) identifying a subset as a prospective binding site subset if the subset meets one or more criteria. This method may further include the step of assigning to a prospective binding site subset one or more descriptors, and may also further include the step of identifying the position of a candidate molecule relative to the target molecule based on the value or values of the descriptors assigned to a prospective binding site subset. In versions of these method, in step (c) the set of points may be grouped into one or more subsets of points in which all points in a subset are contiguous with other points in the subset, or are contiguous with other points in the subset and a point is included in the subset only if all of the points within a cutoff distance of the point are in the set of points identified in step (b). In another version of this method, in step (d) a subset meets the one or more criteria if the number of points in the subset exceeds a cutoff number.

In all of the methods described in this patent that include determination of a spatial extent surface, the spatial extent surface may be determined using a geometric property of the target molecule, which in various versions may be the van der Waals spheres of the atoms comprising the target molecule, the position of the atoms comprising the target molecule, or the solvent accessible surface of the target molecule. In specific versions of the methods, the spatial extent surface may be a convex hull surface, an alpha surface, or an ellipsoid.

In all of the methods described in this patent that include determination of a convex hull, the convex hull may be determined based on a set of points characteristic of the spatial extent of the target molecule, and in one version the set of points characteristic of the spatial extent of the target molecule may be the set of positions of one or more of the atoms comprising the target molecule.

In all of the methods described in this patent that include determination of an enclosed volume that is outside of the target molecule, the enclosed volume may be determined to be outside of the target molecule if (i) for each atom comprising the target molecule the distance from points in the enclosed volume to the atom is greater than the van der Waals radius of the atom, (ii) the enclosed volume is outside of the solvent accessible surface of the target molecule, or (iii) the potential energy at points in the enclosed volume are less than a cutoff energy.

In all of the methods described in this patent that include one or more descriptors characteristic of the spatial extent of an enclosed volume or assigned to a site adjacent to the enclosed volume, the one or more descriptors may include the volume of the enclosed volume, the volume of a subvolume contained within the enclosed volume, and spatial moments of the enclosed volume. In one version, the subvolume contained within the enclosed volume may be a volume of the same overall shape of the enclosed volume but of smaller dimensions. In one version, the one or more descriptors may be the centroid of the enclosed volume and the first principal component of the enclosed volume.

In all of the methods described in this patent that include the step of identifying a position for a candidate molecule relative to the target molecule based on the value or values of the descriptors of an enclosed volume or descriptors assigned to a site adjacent to the enclosed volume, the position of the candidate molecule may be identified such that the center of mass of the candidate molecule is approximately equal to the centroid of the enclosed volume, or the position of the candidate molecule may be identified such that the center of mass of the candidate molecule is equal to or approximately equal to the centroid of the enclosed volume and the first principal component of the mass distribution of the candidate molecule is aligned with or approximately aligned with the first principal component of the enclosed volume.

In one version of the methods described in this patent the target molecule may be a protein molecule.

The methods described in this patent may be implemented in a computer system and may be embodied in a computer readable medium.

### DETAILED DESCRIPTION OF THE INVENTION

The inventor has discovered new methods for characterizing the spatial extent of sites on a target molecule and for identifying prospective binding sites on a target molecule. In this section we describe (1) specific aspects of the methods (2) implementation of the methods, including implementation in a computer system, and (3) general uses of the methods.

Brief Summary of Methods

One specific method that may be used for identifying a prospective binding site on a target molecule includes the steps of (1) determining the convex hull of a target molecule where the convex hull is based on a set of points characteristic of the spatial extent of the target molecule, for example the positions of the atoms in the target molecule; (2) identifying a set of points in 3-D space that are inside the convex hull and outside of the target molecule, where a point is considered outside of the target molecule if it is, for example, not within the van der Waals radius of an atom in the target molecule, or is not within a solvent accessible surface as defined by the Connolly method; and (3) identifying one or more subsets of the set of points identified in step (2), where the one or more subsets of points are chosen to identify a potential binding site, and the method of choosing the subsets of points may be, for example, to include only those points in a subset for which all neighboring points are in the set of points and to include in a subset only those groups of adjacent points. A further step in the method may be identifying a subset as adjacent to a prospective binding site if the subset meets one or more criteria, including, for example, if the number of points in the subset exceeds a cutoff number.

In a further step in the above method, a prospective binding site subset of points may be characterized by a reduced description by calculating one or more characteristics of the subset of points, where the reduced description may be, for example, the centroid and first principal component of the subset of points.

In a further step in the above methods, the reduced description characterizing a prospective binding site may be used to provide the initial position for a candidate molecule for a computer simulation of docking of the candidate molecule to the target molecule.

Based in part on the specific method described above, the inventor has identified other methods for identifying prospective binding sites on a target molecule, characterizing the spatial extent of sites on a target molecule, and determining a position of a candidate molecule relative to a target molecule as a step in a computer simulation.

These more general methods include a method for characterizing the spatial extent of a site or sites on a target molecule, including the steps of (a) determining a spatial extent surface characteristic of the overall spatial extent of at least a portion of the target molecule, (b) identifying one or more enclosed volumes meeting a set of criteria comprising (i) being contained within the spatial extent surface, and (ii) being outside of the target molecule, (c) assigning the enclosed volume one or more descriptors characteristic of the spatial extent of the enclosed volume, and (d) characterizing the spatial extent of a site on the target molecule by assigning to the site the one or more descriptors assigned to the enclosed volume adjacent to the site. The more general methods also include a method for identifying a prospective binding site or prospective binding sites on a target molecule, including the steps of (a) through (c) as described in the previous method and step (d) of identifying the site as a prospective binding site if the one or more descriptors assigned to the enclosed volume adjacent to the site meet one or more criteria. The more general methods also include a method for determining a position of a candidate molecule relative to a target molecule as a step in a computer simulation, including the steps of (a) identifying a prospective binding site on a target molecule using any of the methods described in this patent; (b) assigning to the prospective binding site one or more descriptors characteristic of the spatial or electronic extent of the prospective binding site; and (c) determining the position of the candidate molecule relative to the target molecule, where the position of the candidate molecule is determined by the one or more descriptors assigned to the prospective binding site.

In the following sections we describe in detail various aspects of these methods including the following:

(1) target molecules and candidate molecules that may be used;

(2) methods of determining a spatial extent surface characteristic of the overall spatial extent of the target molecule;

(3) methods of identifying an enclosed volume inside the spatial extent surface or meeting the spatial extent criteria and outside of the target molecule;

(4) descriptors characteristic of the enclosed volume;

(5) criteria that may be used to identify a site as a prospective binding site; and

(6) methods for determining a position of a candidate molecule relative to a target molecule.

Target Molecules and Candidate Molecules

The methods described in this patent may be used as part of a procedure to identify candidate molecules that may bind to a target molecule, for example the methods may be used to generate a position for the candidate molecule relative to the target molecule for use in a docking simulation. Generally the target molecule that may be used in the methods described in this patent may be any molecule or molecular fragment for which it is possible to determine a spatial extent surface or one or more criteria characteristic of the overall spatial extent of the molecule. The target molecule may include a complex of one or more molecules or molecular fragments. Target molecules include but are not limited to organic molecules, inorganic molecules, organometalic molecules, neutral molecules, radicals, cations, anions, ionic salts, coordination compounds and molecular fragments of any of the foregoing. Specific target molecules that may be used include but are not limited to proteins, enzymes, and nucleic acids.

Examples of target molecules that are a complex of one or more molecules or molecular fragments include but are not limited to a complex of one or more protein molecules, and a solvated protein molecule.

Many of the methods described in the Background Section are specific to calculations of ligand or small-molecule docking to a protein, and the descriptions thus indicate that the target is a protein. This disclosure in the Background Section is in no way intended to limit the description of target molecule presented in the Summary Section and this Detailed Description Section.

Generally the candidate molecules may be any molecule or molecular fragment. Candidate molecules that may be used include but are not limited to organic molecules, inorganic molecules, organometalic molecules, neutral molecules, radicals, cations, anions, ionic salts, coordination compounds and molecular fragments of any of the foregoing.

Many of the methods described in the Background Section are specific to calculations of ligand or small-molecule docking to a protein, and the descriptions thus indicate that the candidate molecule is a protein ligand. This disclosure in the Background Section is in no way intended to limit the description of candidate molecule presented in the Summary Section and this Detailed Description Section.

Methods for Determining a Spatial Extent Surface Characteristic of the Overall Spatial Extent of the Target Molecule

Some versions of the methods described in this patent include a step of determining a spatial extent surface characteristic of the overall spatial extent of the target molecule. Generally, this spatial extent surface can be any surface characteristic of the overall spatial extent of the target molecule.

In one version of the methods described in this patent, the spatial extent surface may be determined based on some property of the target molecule characteristic of the spatial extent of the target molecule, including but not limited to the spatial distribution of the atoms in the target molecule or the spatial electronic distribution of the atoms and electrons in the target molecule. Other properties that may be used include but are not limited to the collective van der Waals surface of the atoms (i.e., the outer van der Waals surface), a subset of the atoms in the target molecule, a set of points representing a sampling of the collective van der Waals surface of the atoms or a subset of the atoms in the target molecule, and an iso-potential surface or a sampling of points on an iso-potential surface, where the iso-potential surface is a surface of equal potential energy in the potential energy field of the target molecule. In one version of the methods described in this patent, the spatial extent surface is determined based on the spatial distribution of the atoms or a subset of the atoms in the target molecule.

Examples of spatial extent surfaces that can be used to characterize the overall spatial extent of the target molecule include but are not limited to the following:

(1) a convex hull surface calculated for a set of points characteristic of the spatial extent of the target molecule including but not limited to a set of points comprising the spatial positions of the atoms in the target molecule and a set of points comprising spatial positions of iso electronic potential;

(2) an ellipsoid with principal axes proportional to the first principal components of the mass or electronic distributions of the target molecule and center equal to the zeroth order moment of the mass or electronic distributions; and

(3) an alpha surface calculated for a set of points characteristic of the spatial extent of the target molecule, where an alpha surface is a generalization of a convex hull, as described in Edelsbrunner et al. (Edelsbrunner, H, Mucke, E., Three-dimensional alpha shapes, ACM Transactions on Graphics 13, 43-72).

In a version in which the spatial extent surface is a convex hull surface the convex hull surface may be found using any method capable of finding the convex hull of a set of points in 3-D space, including but not limited to the method of Badel-Chagmon et al. and methods described in Skiena, S., The Algorithm Design Manual, Springer-Telos, 1998. The set of points for which the convex hull may be calculated may generally be any set of points characteristic of the spatial extent of the target molecule including but not limited to a set of points comprising the positions of the atoms or a subset of atoms in the target molecule, a set of points comprising points on the surface of van der Waals spheres around each atom in the target molecule or each atom on the surface of the target molecule, and a set of points comprising points on a solvent-accessible surface of the target molecule. In one version of the methods described in this patent, the convex hull is calculated based on the positions of the atoms in the target molecule. This has the effect of reducing the size of the spatial extent surface slightly as compared to one calculated for example based on the van der Waals spheres. As will be described below, such as choice may have the effect of focusing the results on sites in the target molecule that are somewhat deeper.

In the methods described in this patent which require the positions of one or more of the atoms in the target or candidate molecules, the spatial distribution of atoms may be obtained by any method including but not limited to X-ray crystallography, nuclear magnetic resonance (NMR) analysis, computational modeling, or structure data banks, such as the Protein Data Bank (PDB). The atomic coordinates may be expressed in any 3-D coordinate system. Additionally, in particular versions including but not limited to versions in which the target molecule is a protein, the coordinates of the target molecule may include or be modified by the presence of coenzymes, metal ions, glycosyl moieties, solvation shell water molecules, and other small organic or inorganic molecules that are important to determining target molecule structure and function.

When the target molecule is a protein, the positions of hydrogen atoms in the target molecule may not be determined by experimental methods such as X-ray crystallography and NMR, nor are hydrogen coordinates generally available from protein structure data banks such as PDB. Accordingly, the atomic coordinates of a hydrogen atom may be estimated based upon the atomic coordinates of the amino acid residues to which a hydrogen atom is associated.

In the methods described in this patent, a spatial extent surface may be calculated for a portion of the target molecule, and in such cases the methods will characterize or identify as prospective binding sites those sites on the target molecule that are included in the portion of the target molecule used for calculating the spatial extent surface.

Identifying an Enclosed Volume Inside the Spatial Extent Surface and Outside of the Target Molecule

Some of the methods described in this patent include the step of identifying an enclosed volume that is contained within the spatial extent surface and is outside of the target molecule.

Generally, any method capable of identifying a volume meeting the above criteria may be used. One method that may be used is a method for sampling the points in space around and within the target molecule in order to find all points within the one or more enclosed volumes that are not within the target molecule. For each sampled point, it is determined whether the point is inside the spatial extent surface or not. If it is inside, then it is then further determined if it is outside the target molecule. If it is also outside the target molecule, it is added to the set of points meeting the criteria. In one specific nonlimiting example of such a method, a grid is defined at intervals to include all of the space inside a box bounded by the minimum and maximum x, y, and z values of all atoms of the target molecule. The grid size can vary, for example it can be 0.1 to 1 angstrom, or optionally 0.5 angstroms. Any point outside this box cannot be inside the space defined by the spatial extent surface characteristic and does not therefore need to be considered in the sampling.

Determining Points Inside the Spatial Extent Surface

When using methods for identifying an enclosed volume based on points in space, determining if the points in space are inside the spatial extent surface may generally be performed using any method capable of determining if the points are inside the spatial extent surface. When the spatial extent surface is a convex hull, such methods that may be used include but are not limited to a method in which the convex hull surface is represented by a set of half-spaces, pointed in the direction of the convex hull centroid. The points that are inside the hull are those that are in all of the half-spaces. If the half space is represented as a point P on the plane (face of the hull polyhedron) and a direction vector D that is perpendicular to the face, then determination of membership in the half space of an arbitrary point X is determined by comparing the dot product of (X−P) and D. If the value is greater than or equal to zero, point X is in the half space.

Determining the Points Not Inside the Target Molecule

When using methods for identifying an enclosed volume based on points in space, determining if the points in space are outside of the target molecule may generally be performed using any method capable of determining if the points are outside of the target molecule.

In one version of the methods, all points that have a calculated potential energy below some cutoff energy are points outside of the target molecule. Example of potential energies that may be used include but are not limited to the steric energy which can be calculated using Leonard-Jones 6-12 potential or other potential. Typically the probe atom used is a carbon atom, but any atom could be used. In one example, if the energy of the probe atom at the given point is positive, the point is considered inside the target molecule.

In one version of the methods, a point is outside of the target molecule if, for each atom comprising the target molecule or for each atom on the surface or a portion of the surface of the target molecule, the distance from the point to the atom is greater than the van der Waals radius of the atom.

In one version of the methods, a point is outside of the target molecule if it is outside of the solvent accessible surface of the target molecule. The solvent accessible surface may be calculated for example by the Connolly method or by any other method known in the art, including for example methods reviewed in the article at http://www.netsci.org/Science/Compchem/feature14.html.

Using a Criterion or Criteria Characteristic of the Overall Spatial Extent Rather than a Spatial Extent Surface

In one version of the methods described in this patent, an enclosed volume consists of the set of points that meet one or more criteria characteristic of the overall spatial extent of the target molecule and are outside the target molecule. Generally any criterion or criteria characteristic of the overall spatial extent of the target molecule may be used, including but not limited to the criterion of the point being within a spatial extent surface as described above, or the point being within some volume characteristic of the overall spatial extent of the target molecule.

Processing the Enclosed Volume

In some methods described in this patent, an enclosed volume identified by one of the methods described elsewhere in this patent may be further processed to assist in identifying those enclosed volumes of particular interest. For example, processing may assist in identifying those enclosed volumes characteristic of prospective binding sites on the target molecule.

In one version of the methods described in this patent, the enclosed volume may be reduced in size by eliminating an outside shell of the enclosed volume to a certain depth. When the enclosed volume is characterized by a set of points, this version of the method may be implemented by retaining only those points in the enclosed volume for which all neighboring points are also in the enclosed volume. This reduction can be applied iteratively to give the reduced sets of points that represent only the deepest sites in the target molecule.

Another example of processing that may be carried out on an enclosed volume characterized by a set of points is that a set of contiguous points within each enclosed volume is determined by taking one point in the selected set, and including it and its neighbors that are also in the set. This may be done recursively, so that when a neighbor is included, all of its neighbors are also included. Contiguous sets that have fewer than a certain number of member points can be disregarded.

Another example of processing that may be carried out is that only enclosed volumes with a volume greater than a cutoff value are included; when an enclosed volume is characterized by a set of points the processing may include only those volumes that contain a number of points greater than some cutoff number.

Descriptors Characteristic of an Enclosed Volume

An enclosed volume identified using any of the methods described in this patent may be characterized using one or more descriptors. The enclosed volume to be characterized may also be an enclosed volume that has been processed using any of the processing methods described in this patent.

Generally, any reduced description characteristic of the enclosed volume may be used, including but not limited to the volume of the enclosed volume, the spatial moments of the enclosed volume including but not limited to the centroid and first principal components, and the number of spatial grid points contained in the volume. In this patent we use the term “first principal component” to mean the direction vector along which most of the variance in a set of points occurs. This is also described as the eigen vector associated with the largest eigen value of the covariance matrix of the set of points. We describe below one method for determining the first principle component.

Other descriptors that may be used include but are not limited to the position and size of a set of spheres characteristic of the shape of the enclosed volume (for example, the sphere representation used in the DOCK programs described in the background section), and generally any descriptors used in characterizing the spatial or electronic extent of sites on a target molecule, including but not limited to those characterizations used by methods described in the background section.

Generally the centroid and first principal component can be calculated using any method known in the art. When the enclosed volume is characterized by a set of points, the centroid of the set of points and a vector representing the first principal component of the set of points may be calculated, for example, using the following method. The first principle component can be determined using the covariance matrix of the point coordinates. This covariance matrix is a 3 by 3 matrix calculated by multiplying the 3 by N matrix of point coordinates by it own transpose. The vector from the centroid to the most distant point of the set is taken as a starting point, and is iteratively: 1) multiplied by the covariance matrix and 2) normalized to unit length. These iterations stop when the direction of the product vector is no longer changing much. This method of determining the first eigen vector of the covariance matrix (and thus the first principle component) can fail in certain degenerate cases. In these cases, the site shape may have no obvious long axis.

In one version of the methods described in this patent, the descriptors may include more than the centroid and first principal component characterizing the enclosed volume. This method may be used in situations including but not limited to when an enclosed volume is very large. In this version of the methods, the enclosed volume can be characterized by calculating centroids and first principal components for points in addition to the point at the centroid of the enclosed volume. To do this, the points within a cutoff distance of the centroid are deleted. The remaining points are divided into one or more contiguous groups of points. Those groups that have fewer points than a cutoff value may be deleted. The remaining groups are used to generate additional enclosed volume characterizations. In one version of this method, the cutoff that can be used includes but is not limited to a cutoff between 1-20 angstroms, or a cutoff of eight angstroms. Using this version of the methods, contiguous sets of points are identified that have a least a minimum number of points, and the centroid and first principle component of these additional sets of site points can be calculated.

Criterion or Criteria that May be Used to Identify Potential Binding Sites

Enclosed volumes identified using any of the methods described in this patent may be used to identify potential binding sites. Generally, a site on a target molecule will be identified as a prospective binding site if the enclosed volume adjacent to the site meets one or more criteria indicative of a prospective binding site. Generally, any criterion or criteria indicative of a potential binding site may be used, including but not limited to criterion or criteria indicative of a site of a certain size and depth into which a candidate molecule may bind.

Examples of specific criteria that may be used include but are not limited to criteria in which (1) the enclosed volume is larger than a cutoff volume; (2) the number of grid points in the enclosed volume are larger than a cutoff number; (3) moments calculated for the enclosed volume meet some criterion or criteria, for example the centroid and first principal component meet some criterion or criteria that may be but are not limited to the centroid and first principal component being comparable to the centroid and first principal component of sites that are known to be prospective binding sites.

The criterion or criteria described above may be applied to the enclosed volume initially identified or an enclosed volume after processing as described elsewhere in this patent.

A site on a target molecule may also be identified as a potential binding site if one or more descriptors characteristic of the enclosed volume adjacent to the site meet one or more criteria, which can generally be any criterion or criteria indicative of a potential binding site.

Methods for Determining a Position of a Candidate Molecule Relative to a Target Molecule

Enclosed volumes identified using any of the methods described in this patent may be used to identify a position for placement of a candidate molecule relative to the target molecule for use in a computation involving the candidate molecule and target molecule. Such placement may make the computation more efficient by identifying an initial position that may be closer to a docked position than would be a randomly chosen initial position. Generally, the identified position may be related to some characteristic of the enclosed volume or related to one or more descriptors of the enclosed volume.

Examples of placement of candidate molecules include but are not limited to placing the candidate molecule so that the center of mass is close to or at the centroid of an enclosed volume and the first principal component of the candidate molecule is aligned or close to being aligned or antialigned or close to being anti-aligned with the first principal component of enclosed volume. Further placements that may be used include but are not limited to placing a candidate molecule so that its center of electronic distribution (i.e., zeroth order moment of the electronic distribution) is close to or at a point characteristic of the center of the enclosed volume or characteristic of the electron distribution of the target molecule site adjacent to the enclosed volume and the dipole of the candidate molecule is aligned (or anti-aligned) or close to being aligned or anti-aligned with some vector characteristic of the shape of the enclosed volume or characteristic of the electronic distribution of the target molecule site adjacent to the enclosed volume.

Examples of computational methods for which the initial placement described in the methods in this patent may be useful include but are not limited to the computational methods described in the Background Section of this patent and in patent application titled METHODS FOR IDENTIFYING A MOLECULE THAT MAY BIND TO A TARGET MOLECULE, with inventor John Robert Hurst, filed on Aug. 5, 2002, with attorney docket number 529842000100.

The methods described in this patent may be implemented using any device capable of implementing the methods. Examples of devices that may be used include but are not limited to electronic computational devices, including computers of all types. Examples of computer systems that may be used include but are not limited to a Pentium based desktop computer or server, running Microsoft NT, or a Linux based operating platform such as distributed by Red Hat, Inc., or a suitable operating platform. Such computer systems may be readily purchased from Dell, Inc. (Austin, Tex.), Hewlett-Packard, Inc., (Palo Alto, Calif.) or Compaq Computers, Inc., (Houston, Tex.).

When the methods described in this patent are implemented in a computer, the computer program that may be used to configure the computer to carry out the steps of the methods may be contained in any computer readable medium capable of containing the computer program. Examples of computer readable medium that may be used include but are not limited to diskettes, hard drives, CD-ROMs, DVDs, ROM, RAM, punch cards and other memory and computer storage devices. The computer program that may be used to configure the computer to carry out the steps of the methods may also be provided over an electronic network, for example, over the Internet, world wide web, an intranet, or other network.

In one example, the methods described in this patent may be implemented in a system comprising a processor and a computer readable medium that includes program code means for causing the system to carry out the steps of the methods described in this patent. The processor may be any processor capable of carrying out the operations needed for implementation of the methods. The program code means may be any code that when implemented in the system can cause the system to carry out the steps of the methods described in this patent. Examples of program code means include but are not limited to instructions to carry out the methods described in this patent written in a high level computer language such as C++, Java, or Fortran; instructions to carry out the methods described in this patent written in a low level computer language such as assembly language; or instructions to carry out the methods described in this patent in a computer executable form such as compiled and linked machine language.

The output from an implementation of the methods described in this patent may be embodied in any article of manufacture capable of containing the output and may also be embodied in computer storage or memory devices capable of containing the output. Examples of articles of manufacture that may be used include but are not limited to diskettes, hard drives, CD ROMs and DVDs. Output from implementation of the methods described in this patent may also be provided over an electronic network, for example, over the Internet, world wide web, an intranet, or other network. A user of the information output from the methods described in this patent may access the information through any device capable of transmitting the information to the user, including but not limited to computer monitors, CRTs, telephones, projection devices, paper images and text. A person becoming aware of the information generated as an output from the methods described in this patent may use the information in a variety of ways including the uses described in the next section and uses described elsewhere in this patent. A person may become aware of the information generated as an output from the methods described in this patent in a variety of ways including but not limited to accessing the information via a computer network, accessing the information telephonically, and accessing the information in written form.

Uses of the Methods

The methods described in this patent may be used in a variety of ways including but not limited to identifying prospective binding sites on a target molecule and providing a reduced description of sites on a target molecule including but not limited to a description of the spatial or electronic nature of sites on a target molecule. The identification of prospective binding sites may be useful in computer simulations in which candidate molecules may be placed in or close to the prospective binding sites, and may also be useful in placing candidate molecules close to a prospective binding site as a starting point or 3-D searching methods.

Providing a reduced description of the spatial or electronic nature of sites on a target molecule may be useful for identifying target molecules that may bind to classes of molecules or that may be involved in particular biological pathways, such identification may be possible by comparing the characteristics of sites to characteristics of sites on target molecules known to bind to classes of molecules or known to be involved in particular biological pathways. In addition, providing a reduced description may be useful in methods including but not limited to providing a coarse filtering step in a docking process or as part of the query for a docking process.

The examples described in this patent are for illustrative purposes only and various modifications or changes will be suggested to persons skilled in the art and are to be included within the disclosure in this application and scope of the claims. All publications, patents and patent applications cited in this patent are hereby incorporated by reference in their entirety for all purposes to the same extent as if each individual publication, patent or patent application were specifically and individually indicated to be so incorporated by reference.

What is claimed is:

1. A method for identifying a set of points identifying a prospective binding site on a target molecule, the method comprising the steps of
(a) determining a convex hull surface for a target molecule;
(b) identifying a set of points that are inside the convex hull surface and outside of the target molecule;
(c) grouping the set of points identified in step (b) into one or more subsets; and
(d) identifying a subset as a prospective binding site subset if the subset meets one or more criteria.

2. The method of claim 1, wherein the convex hull surface is determined based on a set of points characteristic of the spatial extent of the target molecule.

3. The method of claim 2, wherein the target molecule comprises one or more atoms and the set of points characteristic of the spatial extent of the target molecule is the set of positions of one or more of the atoms comprising the target molecule.

4. The method of claim 1, wherein the target molecule is a protein molecule.

5. The method of claim 1, wherein the target molecule comprises one or more atoms and in step (b) a point is outside of the target molecule if for each atom comprising the target molecule the distance from the point to the atom is greater than the van der Waals radius of the atom.

6. The method of claim 1, wherein the target molecule has a solvent accessible surface and in step (b) a point is outside of the target molecule if it is outside of the solvent accessible surface.

7. The method of claim 1, wherein in step (b) a point is outside of the target molecule if the potential energy at the point is less than a cutoff energy.

8. The method of claim 1, wherein in step (c) the set of points is grouped into one or more subsets of points in which all points in a subset are contiguous with other points in the subset.

9. The method of claim 8, wherein the set of points is grouped into one or more subsets of points in which all points in a subset are contiguous with other points in the subset and a point is included in the subset only if all of the points within a cutoff distance of the point are in the set of points identified in step (b).

10. The method of claim 1, wherein in step (d) a subset meets the one or more criteria if the number of points in the subset exceeds a cutoff number.

11. The method of claim 1, further comprising the step of assigning to a prospective binding site subset of points one or more descriptors.

12. The method of claim 1 1, further comprising the step of identifying a position for a candidate molecule relative to the target molecule, where the position of the candidate molecule is determined by the value or values of the one or more descriptors.

13. The method of claim 12, wherein the one or more descriptors include the centroid of the prospective binding site subset of points and the position of the candidate molecule is such that the center of mass of the candidate molecule is equal to or approximately equal to the centroid of the prospective binding site subset of points.

14. The method of claim 13, wherein the one or more descriptors further include the first principal component of the prospective binding site subset of points and the position of the candidate molecule is such that the center of mass of the candidate molecule is equal to or approximately equal to the centroid of the prospective binding site subset of points and the first principal component of the mass distribution of the candidate molecule is aligned or approximately aligned with the first principal component of the prospective binding site subset of points.

15. A method for characterizing a spatial extent of a volume adjacent to a site on a target molecule, where the target molecule has an overall spatial extent, the method comprising the steps of:
(a) determining a spatial extent surface characteristic of the overall spatial extent of at least a portion of the target molecule;
(b) identifying a set of one or more enclosed volumes each enclosed volume in the set meeting a set of criteria comprising (i) being contained within the spatial extent surface, and (ii) being outside of the target molecule; and
(c) assigning to one or more of the enclosed volumes in the set of one or more enclosed volumes one or more descriptors characteristic of the spatial extent of the enclosed volume.

16. The method of claim 15, wherein the target molecule is a protein.

17. The method of claim 15 wherein the spatial extent surface is determined using a geometric property of the target molecule.

18. The method of claim 17, wherein the target molecule comprises one or more atoms and the geometric property is the van der Waals spheres of the atoms comprising the target molecule.

19. The method of claim 17, wherein the target molecule comprises one or more atoms and the geometric property is the position of the atoms comprising the target molecule.

20. The method of claim 17, wherein the target molecule has a solvent accessible surface and the geometric property is the solvent accessible surface.

21. The method of claim 15, wherein the spatial extent surface is a convex hull surface.

22. The method of claim 15, wherein the spatial extent surface is an alpha surface.

23. The method of claim 15 wherein the spatial extent surface is an ellipsoid.

24. The method of claim 15, wherein the target molecule comprises one or more atoms and in step (b) an enclosed volume is outside of the target molecule if for each atom comprising the target molecule the distance from points in the enclosed volume to the atom is greater than the van der Waals radius of the atom.

25. The method of claim 15, wherein the target molecule has a solvent accessible surface and in step (b) an enclosed volume is outside of the target molecule if the enclosed volume is outside of the solvent accessible surface.

26. The method of claim 15, wherein in step (b) an enclosed volume is outside of the target molecule if the potential energy at points in the enclosed volume is less than a cutoff energy.

27. The method of claim 15, wherein the one or more descriptors characteristic of the spatial extent of an enclosed volume in the set of one or more enclosed volumes include one or more descriptors selected from the group consisting of the volume of the enclosed volume, the volume of a subvolume contained within the enclosed volume, and spatial moments of the enclosed volume.

28. The method of claim 27, wherein the subvolume contained within the enclosed volume is a volume of the same overall shape of the enclosed volume but of smaller dimensions.

29. The method of claim 15, wherein the one or more descriptors characteristic of the spatial extent of an enclosed volume in the set of one or more enclosed volumes include one or more descriptors selected from the group consisting of the centroid of the enclosed volume and the first principal component of the enclosed volume.

30. The method of claim 15, further comprising the step of identifying a position for a candidate molecule relative to the target molecule, where the position of the candidate molecule is determined by the value of the one or more descriptors associated with an enclosed volume in the set of one or more enclosed volumes.

31. The method of claim 30, wherein the one or more descriptors include the centroid of the enclosed volume adjacent to the site and the position of the candidate molecule is such that the center of mass of the candidate molecule is approximately equal to the centroid of the enclosed volume.

32. The method of claim 31, wherein the one or more descriptors further include the first principal component of the enclosed volume adjacent to the site and the position of the candidate molecule is such that the center of mass of the candidate molecule is equal to or approximately equal to the centroid of the enclosed volume and the first principal component of the mass distribution of the candidate molecule is aligned with or approximately aligned with the first principal component of the enclosed volume.

33. The method of claim 15, further comprising the step of characterizing the spatial extent of a site on a target molecule by assigning to the site the one or more descriptors assigned to one or more enclosed volumes adjacent to the site.

34. A method for identifying a site on a target molecule as a prospective binding site, where the target molecule has an overall spatial extent, the method comprising the steps of
(a) determining a spatial extent surface characteristic of the overall spatial extent of at least a portion of the target molecule;
(b) identifying a set of one or more enclosed volumes each enclosed volume in the set of one or more enclosed volumes meeting a set of criteria comprising (i) being contained within the spatial extent surface, and (ii) being outside of the target molecule;
(c) assigning to one or more of the enclosed volumes in the set of one or more enclosed volumes one or more descriptors characteristic of the spatial extent of the enclosed volume; and
(d) identifying a site on the target molecule as a prospective binding site if the one or more descriptors assigned to an enclosed volume adjacent to the site meet one or more criteria.

35. The method of claim 34, wherein the target molecule is a protein.

36. The method of claim 34 wherein the spatial extent surface is determined using a geometric property of the target molecule.

37. The method of claim 36, wherein the target molecule comprises one or more atoms and the geometric property is the van der Waals spheres of the atoms comprising the target molecule.

38. The method of claim 36, wherein the target molecule comprises one or more atoms and the geometric property is the position of the atoms comprising the target molecule.

39. The method of claim 36, wherein the target molecule has a solvent accessible surface and the geometric property is the solvent accessible surface.

40. The method of claim 36, wherein the spatial extent surface is a convex hull surface.

41. The method of claim 36, wherein the spatial extent surface is an alpha surface.

42. The method of claim 34 wherein the spatial extent surface is an ellipsoid.

43. The method of claim 34, wherein the target molecule comprises one or more atoms and in step (b) an enclosed volume is outside of the target molecule if for each atom comprising the target molecule the distance from points in the enclosed volume to the atom is greater than the van der Waals radius of the atom.

44. The method of claim 34, wherein the target molecule has a solvent accessible surface and in step (b) an enclosed volume is outside of the target molecule if the enclosed volume is outside of the solvent accessible surface.

45. The method of claim 34, wherein in step (b) an enclosed volume is outside of the target molecule if the potential energy at points in the enclosed volume is less than a cutoff energy.

46. The method of claim 34, wherein the one or more descriptors characteristic of the spatial extent of an enclosed volume in the set of one or more enclosed volumes include one or more descriptors selected from the group consisting of the volume of the enclosed volume, the volume of a subvolume contained within the enclosed volume, and spatial moments of the enclosed volume.

47. The method of claim 46, wherein the subvolume contained within the enclosed volume is a volume of the same overall shape of the enclosed volume but of smaller dimensions.

48. The method of claim 34, wherein the one or more descriptors characteristic of the spatial extent of an enclosed volume in the set of one or more enclosed volumes include one or more descriptors selected from the group consisting of the centroid of the enclosed volume and the first principal component of the enclosed volume.

49. The method of claim 34, wherein in step (c) one of the descriptors assigned to an enclosed volume in the set of one or more enclosed volumes is a volume descriptor and in step (d) the site is identified as a prospective binding site if the volume descriptor is greater than a cutoff value.

50. The method of claim 49, wherein the volume descriptor is a descriptor characteristic of the volume of the enclosed volume or a descriptor characteristic of the volume of a subvolume of the enclosed volume with the same overall shape as the enclosed volume but of smaller dimensions.

51. The method of claim 34, further comprising the step of identifying a position for a candidate molecule relative to the target molecule, where the position of the candidate molecule is determined by the value of the one or more descriptors associated with a prospective binding site.

52. The method of claim 51, wherein the one or more descriptors include the centroid of the enclosed volume adjacent to the prospective binding site and the position of the candidate molecule is such that the center of mass of the candidate molecule is equal to or approximately equal to the centroid of the enclosed volume.

53. The method of claim 52, wherein the one or more descriptors further include the first principal component of the enclosed volume adjacent to the prospective binding site and the position of the candidate molecule is such that the center of mass of the candidate molecule is equal to or approximately equal to the centroid of the enclosed volume and the first principal component of the mass distribution of the candidate molecule is aligned with or approximately aligned with the first principal component of the enclosed volume.

54. A method for identifying a site on a target molecule as a prospective binding site, where the target molecule has an overall spatial extent, the method comprising the steps of
(a) identifying a set of one or more enclosed volumes, each enclosed volume in the set meeting a set of criteria comprising (i) being outside of a target molecule, and (ii) meeting one or more criteria characteristic of the overall spatial extent of the target molecule;
(b) assigning to the enclosed volume one or more descriptors characteristic of the spatial extent of the enclosed volume; and
(c) identifying a site on the target molecule as a prospective binding site if the one or more descriptors assigned to an enclosed volume adjacent to the site meet one or more criteria.

55. The method of claim 54, wherein the target molecule is a protein.

56. The method of claim 54, wherein the target molecule comprises one or more atoms and in step (a) an enclosed volume is outside of the target molecule if points in the enclosed volume meet one or more criteria selected from the group consisting of (1) for each atom comprising the target molecule the distance from the point to the atom is greater than the van der Waals radius of the atom; (2) the target molecule has a solvent accessible surface and the point is outside of the solvent accessible surface; and (3) the potential energy at the point is less than a cutoff energy.

57. The method of claim 54, wherein in step (a) an enclosed volume meets the one or more criteria characteristic of the overall spatial extent of the target molecule if points in the enclosed volume are within a surface characteristic of the overall spatial extent of the target molecule.

58. The method of claim 57, wherein the surface characteristic of the overall spatial extent of the target molecule is a convex hull surface calculated for a set of points characteristic of the overall spatial extent of the target molecule.

59. The method of claim 57, wherein the surface characteristic of the overall spatial extent of the target molecule is an alpha surface.

60. The method of claim 54, wherein the one or more descriptors characteristic of the spatial extent of an enclosed volume include one or more descriptors selected from the group consisting of the volume of the enclosed volume, the volume of a subvolume contained within the enclosed volume, and spatial moments of the enclosed volume.

61. The method of claim 60, wherein the subvolume contained within the enclosed volume is a volume of the same overall shape of the enclosed volume but of smaller dimensions.

62. The method of claim 54, wherein the one or more descriptors characteristic of the spatial extent of an enclosed volume in the set of one or more enclosed volumes include one or more descriptors selected from the group consisting of the centroid of the enclosed volume and the first principal component of the enclosed volume.

63. The method of claim 54, wherein in step (b) one of the descriptors assigned to an enclosed volume is a volume descriptor, and in step (c) the site is identified as a prospective binding site if the value of the volume descriptor is greater than a cutoff value.

64. The method of claim 63, wherein the volume descriptor is a descriptor characteristic of the volume of the enclosed volume or a descriptor characteristic of the volume of a subvolume of the enclosed volume with the same overall shape as the enclosed volume but of smaller dimensions.

65. The method of claim 54, further comprising the step of identifying a position for a candidate molecule relative to the target molecule, where the position of the candidate molecule is determined by the value of the one or more descriptors associated with a prospective binding site.

66. The method of claim 65, wherein the one or more descriptors include the centroid of the enclosed volume adjacent to the prospective binding site and the position of the candidate molecule is such that the center of mass of the candidate molecule is equal to or approximately equal to the centroid of the enclosed volume.

67. The method of claim 66, wherein the one or more descriptors further include the first principal component of the enclosed volume adjacent to the prospective binding site and the position of the candidate molecule is such that the center of mass of the candidate molecule is equal to or approximately equal to the centroid of the enclosed volume and the first principal component of the mass distribution of the candidate molecule is aligned with or approximately aligned with the first principal component of the enclosed volume.

68. A method for determining a position of a candidate molecule relative to a target molecule for use in a computer simulation, the method comprising the steps of
(a) identifying a prospective binding site on a target molecule, where the prospective binding site is characterized by one or more descriptors; and
(b) identifying a position for a candidate molecule relative to the target molecule, where the position of the candidate molecule is determined by the value or values of the one or more descriptors.

69. The method of claim 68, wherein the prospective binding site is identified according to the method in claim 34.

70. The method of claim 68, wherein the prospective binding site is identified according to the method in claim 54.

71. The method of claim 68, wherein the one or more descriptors include the centroid of an enclosed volume adjacent to the prospective binding site and the position of the candidate molecule is such that the center of mass of the candidate molecule is equal to or approximately equal to the centroid of the enclosed volume.

72. The method of claim 71, wherein the one or more descriptors further include the first principal component of the enclosed volume adjacent to the prospective binding site and the position of the candidate molecule is such that the center of mass of the candidate molecule is equal to or approximately equal to the centroid of the enclosed volume and the first principal component of the mass distribution of the candidate molecule is aligned with or approximately aligned with the first principal component of the enclosed volume.

73. A system for identifying a set of points identifying a prospective binding site on a target molecule, the system comprising:
(I) a processor; and
(II) a computer readable medium having computer readable program code means embodied therein for causing the system to identify a set of points identifying a prospective binding site on a target molecule, the computer readable program code means comprising: (a) a computer readable program code means for causing a computer to carry out the step of determining a convex hull surface for a target molecule; (b) a computer readable program code means for causing a computer to carry out the step of identifying a set of points that are inside the convex hull surface and outside of the target molecule; (c) a computer readable program code means for causing a computer to carry out the step of grouping the set of points identified in step (b) into one or more subsets; and (d) a computer readable program code means for causing a computer to carry out the step of identifying a subset as a prospective binding site subset if the subset meets one or more criteria.

74. An article of manufacture comprising a computer useable medium having computer readable program code means embodied therein for identifying a set of points identifying a prospective binding site on a target molecule, the computer readable program code means comprising: (a) a computer readable program code means for causing a computer to carry out the step of determining a convex hull surface for a target molecule; (b) a computer readable program code means for causing a computer to carry out the step of identifying a set of points that are inside the convex hull surface and outside of the target molecule; (c) a computer readable program code means for causing a computer to carry out the step of grouping the set of points identified in step (b) into one or more subsets; and (d) a computer readable program code means for causing a computer to carry out the step of identifying a subset as a prospective binding site subset if the subset meets one or more criteria.

75. A system for characterizing a spatial extent of a volume adjacent to a site on a target molecule, the system comprising:
(I) a processor; and
(II) a computer readable medium having computer readable program code means embodied therein for causing the system to characterize a spatial extent of a volume adjacent to a site on a target molecule, where the target molecule has an overall spatial extent, the computer readable program code means comprising: (a) a computer readable program code means for causing a computer to carry out the step of determining a spatial extent surface characteristic of the overall spatial extent of at least a portion of the target molecule; (b) a computer readable program code means for causing a computer to carry out the step of identifying a set of one or more enclosed volumes each enclosed volume in the set meeting a set of criteria comprising (i) being contained within the spatial extent surface, and (ii) being outside of the target molecule; and (c) a computer readable program code means for causing a computer to carry out the step of assigning to one or more of the enclosed volumes in the set of one or more enclosed volumes one or more descriptors characteristic of the spatial extent of the enclosed volume.

76. An article of manufacture comprising a computer useable medium having computer readable program code means embodied therein for characterizing a spatial extent of a volume adjacent to a site on a target molecule, where the target molecule has an overall spatial extent, the computer readable program code means comprising: (a) a computer readable program code means for causing a computer to carry out the step of determining a spatial extent surface characteristic of the overall spatial extent of at least a portion of the target molecule; (b) a computer readable program code means for causing a computer to carry out the step of identifying a set of one or more enclosed volumes each enclosed volume in the set meeting a set of criteria comprising (i) being contained within the spatial extent surface, and (ii) being outside of the target molecule; and (c) a computer readable program code means for causing a computer to carry out the step of assigning to one or more of the enclosed volumes in the set of one or more enclosed volumes one or more descriptors characteristic of the spatial extent of the enclosed volume.

77. A system for identifying a site on a target molecule as a prospective binding site, the system comprising:
(I) a processor; and
(II) a computer readable medium having computer readable program code means embodied therein for causing the system to identify a site on a target molecule as a prospective binding site, where the target molecule has an overall spatial extent, the computer readable program code means comprising: (a) a computer readable program code means for causing a computer to carry out the step of determining a spatial extent surface characteristic of the overall spatial extent of at least a portion of the target molecule; (b) a computer readable program code means for causing a computer to carry out the step of identifying a set of one or more enclosed volumes each enclosed volume in the set of one or more enclosed volumes meeting a set of criteria comprising (i) being contained within the spatial extent surface, and (ii) being outside of the target molecule; (c) a computer readable program code means for causing a computer to carry out the step of assigning to one or more of the enclosed volumes in the set of one or more enclosed volumes one or more descriptors characteristic of the spatial extent of the enclosed volume; and (d) a computer readable program code means for causing a computer to carry out the step of identifying a site on the target molecule as a prospective binding site if the one or more descriptors assigned to an enclosed volume adjacent to the site meet one or more criteria.

78. An article of manufacture comprising a computer useable medium having computer readable program code means embodied therein for identifying a site on a target molecule as a prospective binding site, where the target molecule has an overall spatial extent, the computer readable program code means comprising: (a) a computer readable program code means for causing a computer to carry out the step of determining a spatial extent surface characteristic of the overall spatial extent of at least a portion of the target molecule; (b) a computer readable program code means for causing a computer to carry out the step of identifying a set of one or more enclosed volumes each enclosed volume in the set of one or more enclosed volumes meeting a set of criteria comprising (i) being contained within the spatial extent surface, and (ii) being outside of the target molecule; (c) a computer readable program code means for causing a computer to carry out the step of assigning to one or more of the enclosed volumes in the set of one or more enclosed volumes one or more descriptors characteristic of the spatial extent of the enclosed volume; and (d) a computer readable program code means for causing a computer to carry out the step of identifying a site on the target molecule as a prospective binding site if the one or more descriptors assigned to an enclosed volume adjacent to the site meet one or more criteria.

79. A system for identifying a site on a target molecule as a prospective binding site, the system comprising:
(I) a processor; and
(II) a computer readable medium having computer readable program code means embodied therein for causing the system to identify a site on a target molecule as a prospective binding site, where the target molecule has an overall spatial extent, the computer readable program code means comprising: (a) a computer readable program code means for causing a computer to carry out the step of (a) a computer readable program code means for causing a computer to carry out the step of identifying a set of one or more enclosed volumes, each enclosed volume in the set meeting a set of criteria comprising (i) being outside of a target molecule, and (ii) meeting one or more criteria characteristic of the overall spatial extent of the target molecule; (b) a computer readable program code means for causing a computer to carry out the step of assigning to the enclosed volume one or more descriptors characteristic of the spatial extent of the enclosed volume; and (c) a computer readable program code means for causing a computer to carry out the step of identifying a site on the target molecule as a prospective binding site if the one or more descriptors assigned to an enclosed volume adjacent to the site meet one or more criteria.

80. An article of manufacture comprising a computer useable medium having computer readable program code means embodied therein for identifying a site on a target molecule as a prospective binding site, where the target molecule has an overall spatial extent, the computer readable program code means comprising: (a) a computer readable program code means for causing a computer to carry out the step of identifying a set of one or more enclosed volumes, each enclosed volume in the set meeting a set of criteria comprising (i) being outside of a target molecule, and (ii) meeting one or more criteria characteristic of the overall spatial extent of the target molecule; (b) a computer readable program code means for causing a computer to carry out the step of assigning to the enclosed volume one or more descriptors characteristic of the spatial extent of the enclosed volume; and (c) a computer readable program code means for causing a computer to carry out the step of identifying a site on the target molecule as a prospective binding site if the one or more descriptors assigned to an enclosed volume adjacent to the site meet one or more criteria.
